(19) United States
(12) Patent Application Publication
(10) Pub. No.: US 2005/0118278 A1
(43) Pub. Date: Jun. 2, 2005

(54) Pharmaceutical or cosmetic composition, preventing or reducing itch and local reaction from stings of insects

(76) Inventors: Evgeny Nikolaevich Ananiev, Moscow (RU); Margarita Alexeevna Imasheva, Moscow (RU); Vyacheslav Grigorievich Silaev, Moscow (RU); Evgeny Alexandrovich Fokin, Moscow (RU); Viktor Yakovlevich Shulga, Reutov (RU)

Correspondence Address: Ananiev Evgeny Nikolaevich, Via Campo Gallo 21/10, Areze Milano, 20020, IT

(21) Appl. No.: 10/399,149

(22) PCT Filed: Dec. 9, 2002
(86) PCT No.: PCT/RU02/00526

(51) Int. Cl. A61K 33/24

(52) U.S. Cl. 424/617

(57) ABSTRACT

The present invention relates to medicine, more particularly to pharmaceutical or cosmetic compositions, used for application on the skin, including the region of eyes, as well as on the lips and the mucosa of nasal, oral and laryngeal cavities of the man or the animal, with the purpose of preventing or reducing itch and local reaction from stings of insects, in particular mosquitoes, wasps, horse-flies etc. Local reaction from stings of insects is understood as pain, burning sensation, tissue edema, and hyperemia (redness around the sting site). The pharmaceutical or cosmetic composition, preventing or reducing the itch and the local reaction from the stings of insects, contains soluble salt or mixture of soluble salts of one or several rare earth elements, triethylene glycol, glycerine, polyethylene oxide, ethanol, sodium (potassium) hydroxide and water. When applied on the skin or the mucosa immediately after a sting, the composition leads within several tens of seconds to relief or entire disappearance of the itch, the pain and the burning, prevents edematization and then promotes disappearance of the edema. When the claimed composition is applied preventively, a prophylactic action on preventing or reducing manifestation of the itch and the local reaction from the stings is provided for 1-2 hours after the composition application.

### FIELD OF TECHNIQUE

The present invention relates to medicine, more particularly to pharmaceutical or cosmetic compositions, used for application on skin, including the region of eyes, as well as on lips and mucosa of nasal, oral and laryngeal cavities of the man or the animal, with the purpose of preventing or reducing itch and local reaction from stings of insects, in particular mosquitoes, wasps, horse-flies etc. Local reaction from stings of insects is understood as pain, burning sensation, tissue edema, and hyperemia (redness around the sting site) (M. D. Mashkovsky “Pharmaceuticals”. “Meditsina” Publishers, Moscow. 1984. Vol. 2. P. 163).

### BACKGROUND ART

Known in the art is usage of “Zolotaya Zvezda” Balm at stings of insects. The enepidermic balm is rubbed in the site of a sting. However, it is not recommended for application at the injured skin, pustular diseases etc. The balm may cause allergic reactions due to the presence of considerable quantity of essential oils in its composition. There is evidence of a possibility of concurrent reactions developing at application of the balm (M. D. Mashkovsky “Pharmaceuticals”. “Torsing” Publishers, Kharkov. 1997. Vol. 1. P. 322).

The most similar in action is a composition, which relieves itch and prevents cutaneous edemas arising from stings of insects (Russian Federation patent No. 2178692). The composition contains substances of local anaesthetic action, astringents and substances with antiinflammatory action. In particular, a gel composition (example 2), containing the following components in mass. %, is described:

| | |
|---|---|
| Lauret-9 | 6.00 |
| Tannin acid | 1.50 |
| Dipotassium glycyrrhizinate | 1.00 |
| Carbopol 2984 ® | 0.70 |
| 45% caustic soda | 0.05 |
| Ethanol | 30.00 |
| Glycerine | 10.00 |
| Perfume and preservative | |
| quantum satis | |
| Water | The rest |

The known composition in the form of gel is applied on the skin at sites of insect stings, relieves itch and pain sensations, and reduces edemas.

However, application of the known composition is limited, as its application in the region of eyes, on the lips or the mucosa of nasal, oral and laryngeal cavities, as well as on the injured skin, can cause cauterizing action and/or biodamage because of the considerable content of ethanol (30%) in the composition. Biodamage is understood as the change of the skin condition, which may result in formation of rash, fissures and allergic eruption.

### DISCLOSURE OF THE INVENTION

The main object of the invention consists in developing a pharmaceutical or cosmetic composition, preventing or reducing itch and local reaction from stings of insects.

Simultaneously, in case of excessive application and/or long-term presence on the skin, the composition should render no biodamaging effect and have no smell, in order to avoid allergic reactions and discomfort condition.

The object is attained owing to the fact, that the claimed pharmaceutical or cosmetic composition, preventing or reducing itch and local reaction from stings of insects, containing glycerine, ethanol, sodium (potassium) hydroxide and water, according to the invention additionally contains soluble salt or mixture of soluble salts of one or several rare earth elements, triethylene glycol, polyethylene oxide in the following proportion of components (mass. %):

| | |
|---|---|
| Soluble salt or mixture of soluble salts | 0.4-1.0 |
| of one or several rare earth elements | |
| (REE) recalculated to REE cation | |
| Triethylene glycol | 50.0-60.0 |
| Glicerine | 5.0-12.0 |
| Ethanol | 5.0-10.0 |
| Polyethylene oxide | 10.0-15.0 |
| Sodium (potassium) hydroxide | 0.3-0.5 |
| Purified water | The rest |

When applied on the skin or the mucosa immediately after the insect stings, the claimed composition allows to prevent or reduce the itch and the local reaction, within several tens of seconds leads to relief or entire disappearance of the itch, the pain and the burning, and prevents edematization.

When the claimed composition is applied at the sites of insect stings after occurrence of the itch and the local reaction, localization of their development takes place, and subsequently the itch, the pain and the burning disappear, and the edema resorbs.

When the claimed composition is applied preventively, a prophylactic action on preventing or reducing manifestation of the itch and the local reaction from the stings is provided for 1-2 hours after the composition application.

In case of excessive application and long-term presence on the skin and the mucosa, the claimed composition renders no biodamaging effect and practically has no smell.

When present on the skin and/or the mucosa, the claimed composition does not interfere with hidropoiesis, epithelization of the skin and/or the mucosa, injured as a result of insect stings or for some other reason.

The claimed composition causes no allergic reactions, and can be applied repeatedly.

The presence of glycols in the claimed composition in the specified quantity provides uniformity of the ingredients distribution and considerably improves adhesion of the composition on the skin and the mucosas.

The claimed composition employs polyethylene oxide, which, besides its participation in obtaining the required positive effect, provides the necessary consistency of the composition: liquid or gel-like.

The claimed composition, according to the invention, can be prepared in various forms used for such compositions, for example milk, gel or cream. The said forms can be obtained by changing the quantitative content of polyethylene oxide and water within the claimed proportions.

Chlorides, bromides, nitrates, acetates, formates and other soluble compounds of lanthanum, yttrium, cerium, praseodymium, neodymium, promethium, samarium, europium, gadolinium, terbium, dysprosium, holmium, erbium, thulium, ytterbium and lutecium are used as the REE soluble salt.

### BEST VARIANT OF CARRYING OUT THE INVENTION

The technology of preparing the claimed composition is simple and does not differ from technologies of preparing similar compositions. The technology implies mechanical mixing of all components of the composition.

Storage time of the declared composition is 5 years.

The invention is illustrated by examples. The components, given in the examples, are represented in mass. %.

### EXAMPLE 1

(Cream)

| | |
|---|---|
| Hexahydrate of lanthanum nitrate | 0.5 |
| recalculated to lanthanum | |
| Triethylene glycol | 60.0 |
| Glycerine | 7.0 |
| Ethanol | 8.0 |
| Polyethylene oxide - 4000 | 15.0 |
| Sodium hydroxide | 0.5 |
| Purified water | The rest |

### EXAMPLE 2

(Gel)

| | |
|---|---|
| Hexahydrate of lanthanum nitrate | 0.6 |
| recalculated to lanthanum | |
| Triethylene glycol | 55.0 |
| Glycerine | 6.0 |
| Ethanol | 7.0 |
| Polyethylene oxide - 4000 | 10.0 |
| Potassium hydroxide | 0.5 |
| Purified water | The rest |

### EXAMPLE 3

(Milk)

| | |
|---|---|
| Hexahydrate of lanthanum nitrate | 0.8 |
| recalculated to lanthanum | |
| Triethylene glycol | 50.0 |
| Glycerine | 5.0 |
| Ethanol | 5.0 |
| Polyethylene oxide - 4000 | 10.0 |
| Sodium hydroxide | 0.4 |
| Purified water | The rest |

Further, in the following examples instead of lanthanum compound compounds of other rare earth elements are used in the same proportions.

### Example 1a

The composition according to Example 1 wherein hexahydrate of cerium nitrate is used as REE compound.

### Example 1b

The composition according to Example 1 wherein hexahydrate of neodymiium nitrate is used as REE compound.

### Example 1c

The composition according to Example 1 wherein hexahydrate of praseodymium nitrate is used as REE compound.

### Example 2a

The composition according to Example 2 wherein hexahydrate of cerium nitrate is used as REE compound.

### Example 2b

The composition according to Example 2 wherein hexahydrate of neodymium nitrate is used as REE compound.

### Example 2c

The composition according to Example 2 wherein hexahydrate of praseodymium nitrate is used as REE compound.

### Example 3a

The composition according to Example 3 wherein hexahydrate of cerium nitrate is used as REE compound.

### Example 3b

The composition according to Example 3 wherein hexahydrate of neodymium nitrate is used as REE compound.

### Example 3c

The composition according to Example 3 wherein hexahydrate of praseodymium nitrate is used as REE compound.

Since the rare earth elements have identical properties, the soluble compounds of four REE have been used selectively for preparing the compositions.

Efficiency of the claimed composition has been estimated in laboratory conditions by the standard method in relation to yellow fever mosquitoes (Aedes aegyptii), which received only carbohydrate food, without sex distinction. The criterion of the composition efficiency was the time, passing from the application of the tested compositions on the skin in the region of a sting till the beginning of their action. In the controllable conditions stings of mosquitoes were provoked in volunteers on the dorsal side of forearms. In the check experiment one arm was processed with the claimed composition according to examples 1, 1a, 1b, 1c, 2, 2a, 2b, 2c, 3, 3a, 3b, 3c, and the other arm—placebo. The volunteers estimated the tested agents on the time of the itch and pain disappearance, as well as on the edema and hyperemia formation. In course of the trials the time of disappearance of the local reaction signs from mosquito stings after the application of the tested agents was recorded.

Statistically authentic results of the trials are given in Table 1.

The analysis of the data, given in Table 1, shows sufficient activity of the claimed composition irrespective of its form. When the claimed composition was applied immediately after a sting, the itch either did not occur, or, if occurred, disappeared within several tens of seconds, and the edema did not occur at all. Thus, at timely usage of the composition, i.e. immediately after the sting, no consequences of the sting arise.

The efficiency of the claimed composition has been shown at stings of wasps, bees and horse-flies, which are practically always accompanied by itch, burning, pain and edematization. In view of the difficulty of involving volunteers in such trials, tubes with the composition in the form of cream were distributed among inhabitants of country cottages. Then responses were received on the phone from those, who had undergone the stings. 17 stings of wasps, 5 stings of bees and 13 stings of horse-flies were registered. After the preventive application 6 stings of wasps, 1 sting of a bee and 3 stings of horse-flies were registered. All those, who had undergone the stings, noted prevention or reduction of the itch and the local reaction.

The claimed composition has caused no allergic reactions, nor has it dried the skin, it has easily and evenly spreaded over the skin, has soaked into the skin almost immediately without leaving traces or staining the clothes.

### INDUSTRIAL APPLICABILITY

The claimed pharmaceutical or cosmetic composition, according to the invention, can be prepared in various forms. High efficiency on preventing and reducing the itch and the local reaction from the stings of insects makes the claimed composition promising for wide usage in pharmaceutics, cosmetology and veterinary medicine.

TABLE 1
Composition Samples and Efficiency of Their Action,
when Applied on the Skin Immediately After the Mosquito Stings

| Formula- | | | | | | |
|---|---|---|---|---|---|---|
| tion, | Com- | Signs after mosquito stings | | | | |
| Exam- | position | Pain | Burning | Hyperemia | Itch | Edema |
| ple # | Form | sec | sec | sec | sec | sec |
| 1 | Cream | 10-20 | 10-30 | 60-240 | 20-120 | no |
| 1a | Cream | 10-25 | 10-30 | 60-300 | 30-150 | no |
| 1b | Cream | 15-25 | 10-30 | 60-300 | 50-150 | no |
| 1c | Cream | 15-25 | 10-30 | 60-300 | 45-170 | no |
| 2 | Gel | 5-15 | 10-30 | 60-210 | no | no |
| 2a | Gel | 10-20 | 10-30 | 60-240 | 30-120 | no |
| 2b | Gel | 15-20 | 10-30 | 60-270 | 40-130 | no |
| 2c | Gel | 15-20 | 10-30 | 60-270 | 40-150 | no |
| 3 | Milk | 5-15 | 10-25 | 60-200 | no | no |
| 3a | Milk | 15-20 | 10-25 | 60-210 | no | no |
| 3b | Milk | 15-25 | 10-25 | 60-240 | 15-100 | no |
| 3c | Milk | 15-25 | 10-25 | 60-250 | 25-150 | no |
| Placebo | | 500 | 1000 | more than | more | more |
| | | | | 3600 | than | than |
| | | | | | 3600 | 3600 |

1. Pharmaceutical or cosmetic composition, preventing or reducing itch and local reaction from stings of insects, containing glycerine, ethanol, sodium (potassium) hydroxide and water, according to the invention additionally contains soluble salt or mixture of soluble salts of one or several rare earth elements, triethylene glycol, polyethylene oxide in the following proportion of components (mass. %):

| | |
|---|---|
| Soluble salt or mixture of soluble salts | 0.4-1.0 |
| of one or several rare earth elements | |
| (REE) recalculated to REE cation | |
| Triethylene glycol | 50.0-60.0 |
| Glicerine | 5.0-12.0 |
| Ethanol | 5.0-10.0 |
| Polyethylene oxide | 10.0-15.0 |
| Sodium (potassium) hydroxide | 0.3-0.5 |
| Purified water | The rest |
